(19) United States
(12) Patent Application Publication
(10) Pub. No.: US 2023/0051170 A1
(43) Pub. Date: Feb. 16, 2023

(54) PROCESS FOR KIDNEY CELL MANUFACTURE AND TREATMENT

(71) Applicants: KIDNEYCURE LTD, Tel Aviv (IL); TEL HASHOMER MEDICAL RESEARCH INFRASTRUCTURE AND SERVICES LTD, Ramat Gan (IL)

(72) Inventors: Masha SIMANOVSKY, Mazkeret Batya (IL); Benjamin DEKEL, Tel Aviv (IL)

(73) Assignees: KIDNEYCURE LTD, Tel Aviv (IL); TEL HASHOMER MEDICAL RESEARCH INFRASTRUCTURE AND SERVICES LTD, Ramat Gan (IL)

(21) Appl. No.: 17/795,270

(22) PCT Filed: Jan. 25, 2021
(86) PCT No.: PCT/IL2021/050082

§ 371 Date: Jul. 26, 2022

Related U.S. Application Data
(60) Provisional application No. 62/966,584, filed on Jan. 28, 2020.

(51) Int. Cl. C12N 5/071; A61K 35/22; A61P 13/12; A61K 9/00

(52) U.S. Cl. CPC C12N 5/0686; A61K 35/22; A61P 13/12; A61K 9/0019; C12N 2509/00; C12N 2513/00; C12N 2533/90; C12N 2501/113; C12N 2501/125; C12N 2501/11

(57) ABSTRACT

Embodiments described herein relate to a method for preparing cultured cells, the method comprising: obtaining kidney tissue from a human subject; mechanically dissociating the tissue; subjecting the tissue to enzymatic digestion; incubating the tissue with media in a cell culture plate to form cultured cells.

### CROSS REFERENCE TO RELATED APPLICATIONS

Benefit is claimed to U.S. Provisional Patent Application 62/966,584 filed Jan. 28, 2020; the contents of which is incorporated by reference herein in its entirety.

### FIELD

Provided herein are improved methods for manufacture of cells which can be used for treating conditions of the kidney.

### BACKGROUND

Chronic kidney disease (CKD) is a condition in which the ability of the human kidney to properly filter the blood decreases. The loss of kidney function is often gradual, taking years to progress to End-stage kidney disease (ESKD), requiring kidney transplant or dialysis in order for a patient to survive.

CKD is more common in people aged 65 years or older. According to current estimates, about 15% of US adults are estimated to have CKD. Risk factors for CKD include high blood pressure and high blood sugar levels. (Centers for Disease Control and Prevention. Chronic Kidney Disease in the United States, 2019. Atlanta, Ga.: US Department of Health and Human Services, Centers for Disease Control and Prevention; 2019.)

PCT application publication WO2011/141914, incorporated herein by reference, relates to methods for using adult human kidney epithelial cells to manufacture spheroids, capable of generating kidney tubular structures. It has been proposed that these spheroids, when administered to humans in need thereof such as patients suffering from CKD, can generate tubular structures, thereby restoring kidney function.

### SUMMARY

Described herein are improved methods for manufacture of cells which can be used for treating conditions of the kidney, including CKD. The methods of manufacture are advantageous in that the cells used herein may be manufactured by using kidney tissue in small amounts. For example, cells for treatment may be obtained from a kidney biopsy from a healthy individual or a kidney patient, and then those kidney cells may undergo a process which transforms them into cells capable of treating CKD. Alternatively, cultured kidney cells grown the processes described herein may be used to form, for example extracellular vesicles which may be then used for treating a kidney patient.

Embodiments described herein relate to a method for preparing cultured cells, the method comprising: obtaining kidney tissue from a human subject; mechanically dissociating the tissue; subjecting the tissue to enzymatic digestion; incubating the tissue with media in a cell culture plate to form cultured cells.

Processes for obtaining cells from liver, culturing the cells and using said cells as an alternative to liver transplantation have been described. Iansante, V., Mitry, R., Filippi, C. et al. Human hepatocyte transplantation for liver disease: current status and future perspectives. Pediatr Res 83, 232-240 (2018). It is suggested that similar processes may be adapted for use with regard to cultured kidney cells in order to treat kidney disease.

According to an embodiment, upon obtaining human kidney tissue and following a pre-treatment of the tissue, the cells are expanded in a two dimensional (2D) process, then optionally further grown in three dimensional (3D) conditions, then isolated, in order to be used for treatment of CKD.

The pre-treatment of cells comprises a step of mechanically dissociating the tissue. Optionally, the mechanical dissociation may be performed by scratching and/or puncturing the tissue. Optionally, the scratching and/or puncturing still leaves each piece of tissue intact, but potentially increases the surface area of the tissue. Optionally, the tissue then undergoes an enzymatic digestion system. The resulting cells formed are advantageous in that they are higher in number than those formed using previously known methods. Due to the vastly increased yield, less tissue may be taken from a healthy donor or from a CKD patient to provide the same number of therapeutic kidney cells which would only be obtainable from large amounts of tissue using previously known methods.

The foregoing and other objects, features, and advantages will become more apparent from the following detailed description, which proceeds with reference to the accompanying figures.

### BRIEF DESCRIPTION OF THE DRAWINGS

FIG. 1 is a flowchart depicting a method for manufacture of cells, capable of being used for treatment of kidney disease such as CKD; and

FIG. 2 is a flowchart depicting a method for preparation of cellular nephroforms capable of being used for treatment of kidney disease such as CKD.

### DETAILED DESCRIPTION

I. Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Nephroform: A cellular cluster having a generally spherical, or oval shape or a “cactus”-like shape in which irregularly shaped multicellular structures offshoot from a central cell cluster, and having a surface area in the range of between 10000 nm² and 40000 nm². Surface area may be determined by staining of the cells using (5(6)-Carboxyfluorescein N-hydroxysuccinimidyl ester (CFSE) cell labeling kit and calculating surface area with ImageJ software.

II. Overview of Several Embodiments Provided herein are methods for manufacture of cells which can be used in treatment of humans suffering from diseases of the kidney such as CKD. These cells may be designated herein as therapeutic cells. These therapeutic cells which can be used in treatment of humans suffering from diseases of the kidney may be in the form of nephroforms. Also provided herein are methods for treatment, comprising obtaining kidney tissue from a subject, processing the kidney tissue to form a plurality of therapeutic cells, and introducing the therapeutic cells into a kidney of a patient in need thereof.

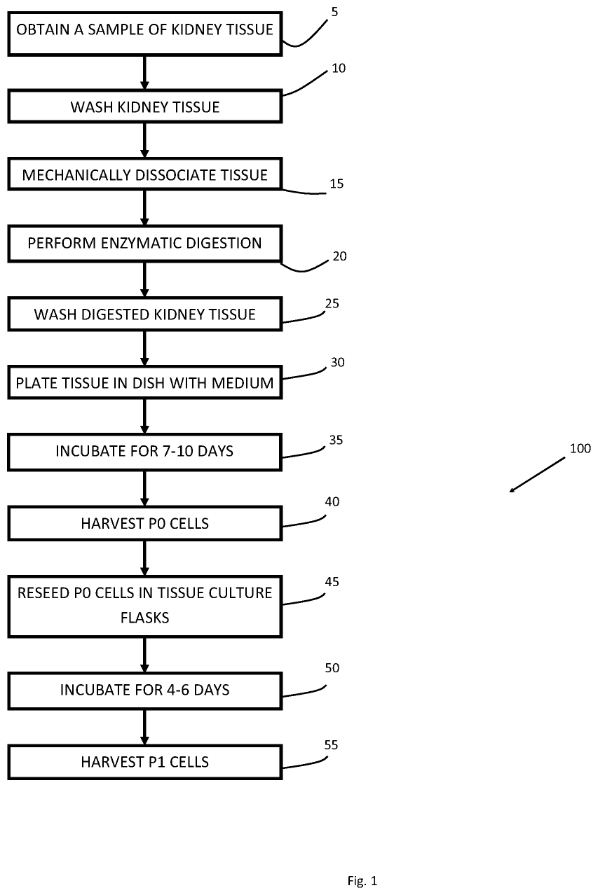

Reference is made to FIG. 1, which is a flowchart depicting a method 100 for preparation of epithelial kidney derived cells capable of being used for treatment of kidney disease such as CKD.

Method 100 comprises block 5, obtaining a sample of kidney tissue. Optionally, the kidney tissue is from a patient suffering from a kidney disease in need of treatment. Optionally, the kidney tissue is from a healthy donor. Optionally, the kidney tissue is obtained through a biopsy. The kidney tissue may be in an amount of between 2 mg and 100 mg. Preferably, the kidney tissue is in an amount of between 2 mg and 50 mg. Alternatively, the tissue may be obtained in pieces ranging in size from between 2 mg and 100 mg or 2 mg and 50 mg.

Method 100 further comprises a block 10, washing kidney tissue. The kidney tissue may be washed with an isotonic solution. Optionally, the isotonic solution contains a salt and glucose. Optionally, the solution is Hank's balanced salt solution (HBSS). Optionally, the solution is free of calcium and magnesium salts. Optionally, Phosphate Bufferred Saline (PBS) without calcium/magnesium is used.

Method 100 further comprises a block 15, in which washed kidney tissue is mechanically dissociated. Optionally, the tissue is scratched or punctured, or both scratched and punctured. Optionally, the tissue is mechanically dissociated into parts that remain attached to each other and the tissue size remains the same after mechanical dissociation, while the surface area of the tissue is increased.

Method 100 further comprises a block 20, performing enzymatic digestion. Enzymatic digestion optionally is performed by exposing renal tissue to a collagenase. Optionally, the collagenase is a type IV collagenase. Optionally, the renal tissue is exposed to a collagenase for between 0.5 hours and 2 hours. Optionally, the renal tissue is exposed to a collagenase at 37° C. Optionally, enzymatic digestion is performed by exposing renal tissue to dispase. Optionally, renal tissue is exposed to both collagenase and dispase. Optionally, after enzymatic digestion, the tissue pieces remain attached to each other and the tissue size remains the same as before enzymatic digestion.

Method 100 further comprises a block 25, washing digested kidney tissue. Optionally, the digested kidney tissue is washed with a buffer solution. Optionally, the solution is phosphate buffered saline solution.

Method 100 further comprises a block 30, plating tissue in a culture dish with the digested kidney tissue, in the presence of medium. The medium may optionally be renal epithelial medium or serum containing medium (SCM). Optionally, the culture dish is a 35 millimeter culture dish, to 100 mm culture dish. Optionally, the weight of the piece of digested kidney tissue is introduced into the culture dish is between 2 mg and 100 mg, preferably between 2 mg and 50 mg.

The SCM optionally comprises fetal bovine serum.

Optionally, the SCM further comprises at least one growth factor. The growth factor may be selected from the group consisting of: Fibroblast growth factor (FGF) Stem cell factor (SCF), and Epidermal growth factor (EGF).

Renal epithelial medium may optionally comprise one or more than one of the following: human Epidermal growth factor (hEGF), transferrin, and insulin.

Method 100 further comprises a block 35, incubating kidney tissue for between 7 and 10 days, preferably 8 days, in the presence of SCM. Optionally, the SCM is replaced once over the course of the incubation. Optionally, the SCM is switched after 2 days. Optionally, the cells are incubated until achieving confluence in the culture dish. The cells formed are designated as passage 0 (P0) cells.

Method 100 further comprises a block 40, harvesting the cultured P0 cells. The cells may be harvested using a dissociating reagent, optionally a trypsin or a trypsin synthetic analogue.

Method 100 further comprises a block 45, reseeding P0 cells in tissue culture flasks. Method 100 further comprises a block 50, incubating cells for 2 to 6 days. Optionally, cells are incubated for 5 days. Optionally, cells are incubated in SCM. The cells after incubation are designated as passage 1 (P1) cells.

Method 100 further comprises a block 55, harvesting P1 cells. Optionally, this harvesting is performed in a manner similar to block 40.

Optionally, additional passages may be performed, to form P2 cells, P3 cells and so on. Preferably, up to 4 passages are performed.

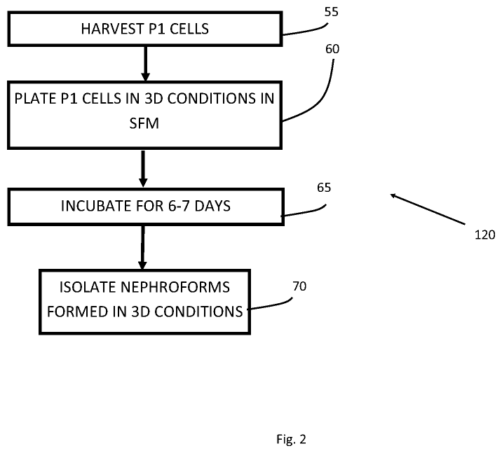

Reference is made to FIG. 2, which is a flowchart depicting a method 120 for preparation of nephroforms from kidney epithelial cells capable of being used for treatment of kidney disease such as CKD.

Method 120 comprises a block 60, plating P1 cells in 3D conditions, in serum free medium (SFM). Optionally, the medium comprises a growth factor selected from the group consisting of epidermal growth factor (EGF) and fibroblast growth factor (FGF). Optionally, the medium comprises insulin and/or progesterone.

Method 120 further comprises a block 65, incubating cells in 3D conditions for 6-7 days. Optionally, the 3D conditions are non-adherent conditions. Optionally, the cells do not attach to the surface of the container in which they are cultured, and a substantial portion of the cells (optionally, above 70% of the cells) can be removed from the surface of the container by mechanical manipulations that do not cause significant damage to the cells.

Method 120 further comprises a block 70, isolating nephroforms formed in 3D conditions.

Optionally, the nephroforms may be then administered to a human in need of treatment.

Nephroforms described herein have been shown to form tubule structures in various in vivo models, indicating that they can be used to promote kidney tubule formation/regeneration in patients in need thereof.

The following examples are provided to illustrate certain particular features and/or embodiments. These examples should not be construed to limit the disclosure to the particular features or embodiments described.

### Examples

Example 1: Preparation of kidney-based cell culture from large amounts of kidney tissue, using a filtration step:

Previous attempts to prepare kidney-based cell culture were initiated by collecting kidney tissue in amounts of greater than 100 mg. Tissue was mixed with collagenase (6 mL) solution for at least 90 minutes while incubating in a shaker at 37° C. The media containing the digested tissue was collected using a pipette and was transferred into a sterile 100 micrometer strainer which was placed on top of a 50 mL tube. The collected media was centrifuged for 5 minutes at 1500 revolutions per minute (rpm) at room temperature. The tissue pellet was resuspended with 1 mL of serum containing media (SCM). The resuspended cells were seeded onto a gelatin coated T-175 flask or uncoated flask, containing 25 mL SCM.

This procedure was not practical for small amounts of tissue, because low yields of cells were obtained when this was attempted.

Example 2: Attempted preparation of kidney-based cell culture from small amounts of kidney tissue using enzymatic digestion:

Small pieces were cut from nonfunctional renal tissue previously washed twice with Hanks' balanced salt solution (HBSS) without Ca and Mg and weighed. Five pieces weighing 13.3 mg, 6.5 mg, 6.3 mg, 13 mg and 16.9 mg each were transferred into separate 1.5 mL Eppendorf tubes containing 1 mL of Collagenase IV solution (1.42 mg/mL Collagenase type IV in Iscove's Modified Dulbecco's Medium (IMDM)). The tubes were incubated for 1 hour at 37° C., 170 rpm. After incubation, enzymatically digested renal tissue was washed with PBS and plated onto 35 mm petri dishes in SCM. SCM was prepared using the following: Iscove's Modified Dulbecco's Medium (IMDM) containing 10% fetal bovine serum (FBS), fibroblast growth factor (FGF) (50 ng/mL), stem cell factor (SCF) (5 ng/mL) and epidermal growth factor (EGF) (50 ng/mL). Only a few cells were observed after one week of cultivation.

Example 3A: Preparation of kidney-based cell culture from small amounts of kidney tissue.

Renal tissue was obtained from a patient suffering from renal cell carcinoma (RCC). The tissue obtained was non-tumor tissue. Small pieces were cut from renal tissue previously washed twice with HBSS without Ca, Mg and were weighed. All pieces were mechanically dissociated by scratching and puncture. Pieces weighing 20 mg and 10 mg were transferred into separate 5 mL round bottom test tubes containing 1 mL of Collagenase IV solution (1.42 mg/mL Collagenase type IV in IMDM). Another piece weighing 8 mg was placed into 1.5 mL Eppendorf tube containing 1 mL of Collagenase IV solution (1.42 mg/mL Collagenase type IV in IMDM). All tubes were incubated for 1 hour at 37° C., 170 rpm. After incubation, enzymatically and mechanically digested renal tissue (EMDRT) was washed with PBS and plated onto 35 mm dishes in 4 mL SCM. After 48 hours, the cells were washed with PBS and SCM was changed. After 7 days in culture, the cells were harvested using TrypLE reagent and counted (Passage 0, P0). All samples were then reseeded into T175 tissue culture flasks and cultured to produce the P1 harvested cells. The cells from the 20 mg piece were cultured for 5 days. The cells originating from the 10 mg piece were cultured for 4 days, and the cells originating from the 8 mg piece were cultured for 3 days.

The cells obtained from each of the kidney tissue samples at P0 and P1 were counted and are tabulated in Table 1 below:

TABLE 1

| | | | Number of P0 cells |
|---|---|---|---|
| | | | per milligram |
| Kidney tissue | P0 cell | P1 cell | starting tissue |
| weight (mg) | number | number | weight |
| 20 | 9 × 10⁵ | 19 × 10⁶ | 4.5 × 10⁴ |
| 10 | 5 × 10⁵ | 13 × 10⁶ | 5 × 10⁴ |
| 8 | 1.4 × 10⁵ | 4 × 10⁶ | 1.75 × 10⁴ |

Example 3B: Preparation of kidney-based cell culture from small amounts of kidney tissue.

Small pieces were cut from non-RCC tissue previously washed twice with HBSS without Ca and Mg and were weighed. All pieces were gently scratched and punctured. Pieces weighing 18.5 mg and 7.8 mg were transferred into separate 5 mL round bottom tubes containing 1 mL of collagenase IV solution (1.42 mg/mL collagenase type IV in IMDM).

Pieces weighing 22.5 mg and 5.6 mg were transferred into separate 5 mL round bottom tubes containing 1 mL of Collagenase-Dispase solution (Dispase 4.0 units/mL containing 300 units/mL of collagenase IV). All tubes were incubated for 1 hour at 37° C., 170 rpm. After incubation tissue pieces were washed with PBS and plated in 35 mm dishes in 1.5 mL SCM. After 48 hours cells were washed with 1 mL PBS and SCM was changed (total volume of 1.5 mL). After 8 days, the cells were harvested with 1 mL TrypLE solution and counted (P0). All samples were then reseeded into T175 tissue culture flasks and cultured for 5 days to produce the P1 harvested cells.

Cell numbers are summarized in Table 2.

TABLE 2

| | | | Number of P0 cells |
|---|---|---|---|
| | | | per milligram |
| Kidney tissue | P0 cell | P1 cell | starting tissue |
| weight (mg) | number | number | weight |
| 18.5 | 4.7 × 10⁶ | 20 × 10⁶ | 2.5 × 10⁵ |
| 7.8 | 1.8 × 10⁶ | 19 × 10⁶ | 2.3 × 10⁵ |
| 22.5 | 5.9 × 10⁶ | 15 × 10⁶ | 2.6 × 10⁵ |
| 5.6 | 1.1 × 10⁶ | 21 × 10⁶ | 2.0 × 10⁵ |

Example 3C: Preparation of kidney-based cell culture from small amounts of kidney tissue.

Small pieces were cut from non-RCC tissue previously washed twice with HBSS without Ca and Mg and were weighed. All pieces were gently scratched and punctured. Pieces weighing 14 mg, 5.7 mg and 2 mg were transferred into separate 5 mL round bottom tubes containing 1 mL of collagenase IV solution (1.42 mg/mL collagenase type IV in IMDM). All tubes were incubated for 1 hour at 37° C., 170 rpm. After incubation tissue pieces were washed with PBS and plated in 35 mm dishes in 2 mL SCM. The 14 mg piece was plated in 100 mm dish in 8 mL SCM. After 48 hours cells were washed with 1 mL PBS and SCM was changed.

After 10 days, the cells were harvested with 1 mL TrypLE solution and counted (P0). All samples were then reseeded into T175 tissue culture flasks and cultured for 5 days to produce the P1 harvested cells.

Cell numbers are summarized in Table 3.

TABLE 3

| | | | Number of P0 cells |
|---|---|---|---|
| | | | per milligram |
| Kidney tissue | P0 cell | P1 cell | starting tissue |
| weight (mg) | number | number | weight |
| 14 | 1.5 × 10⁶ | 19.6 × 10⁶ | 1.1 × 10⁵ |
| 5.7 | 1.24 × 10⁶ | 13.8 × 10⁶ | 2.2 × 10⁵ |
| 2 | 1.62 × 10⁶ | 13 × 10⁶ | 8.1 × 10⁵ |

As can be seen from the examples, novel methods described herein lead to large yields of cells in P0 and P1. As a result, large numbers of therapeutic nephroforms can potentially be manufactured, using small amounts of starting kidney cells. Optionally, cells may be obtained in small pieces, for example, through kidney biopsy, and large yields of nephroforms may still be obtained.

Example 3D: Characterization of cells

Cells prepared in examples 3A-C were analyzed using FACS and/or PCR and/or ELISA and were found to have kidney-related epithelial markers, and certain mesenchymal related markers.

Example 3E: Preparation of kidney-based cell culture from a small amount of kidney tissue originated from needle biopsy

Renal tissue was obtained from one core of needle biopsy from a patient with nephrotic syndrome. The tissue was washed twice with HBSS without Ca and Mg and weighed. A 35 mg fragment was scratched, punctured, and thereafter transferred into a separate 5 mL round bottom tube containing 1 mL of collagenase IV solution (1.42 mg/mL collagenase type IV in IMDM). The tube was incubated for 1 hour at 37° C., 170 rpm. After incubation, the tissue fragment was washed with PBS and plated in 35 mm dishes in 2 mL SCM. After 48 hours cells were washed with 1 mL PBS and SCM was changed. After 8 days, cells were harvested with 1 mL TrypLE solution and counted (P0). Cell count indicated on 1.4×10⁶ cells All cells were then reseeded into T175 tissue culture flasks in 25 mL SCM. After 5 days in culture 16×10⁶ cells (P1) were harvested.

Example 4: Preparation of 3D nephroforms from kidney tissue- based cell culture.

P1 cells originating from a 10 mg piece of kidney tissue were plated in 3D conditions in

SFM, in the amount of 7.5×10⁶ cells per low adhesion flask (1 flask). The nephroforms had average diameter of about 100-300 nm. FACS analysis was performed to characterize the nephroforms. Mesenchymal related markers are reduced relative to the source cells produced in Examples 3A-3C. Epithelial markers are increased relative to the source cells produced in Examples 3A-3C.

Example 5: Treatment of CKD using nephroforms formed from kidney-tissue based cell culture.

Nephroforms are introduced into the human kidney from which the kidney cell tissue originated. The amount of nephroforms comprises cells in an amount between 100 million and 200 million cells. The nephroforms are injected percutaneously, optionally via 5-6 injections, under sonographic/CT guidance into the kidney cortex and/or subcapsular space.

According to an embodiment, described herein are methods for manufacture of cell nephroforms for kidney therapy, the methods comprising: obtaining kidney tissue from a human subject; mechanically dissociating the tissue; subjecting the tissue to enzymatic digestion; incubating the tissue with serum containing media (SCM) having at least one growth factor in a cell culture plate to form cultured cells; harvesting the cultured cells from the cell culture plate using a dissociating agent; and reseeding the cultured cells in tissue culture flasks, and incubating the cells under non-adherent conditions, to form nephroforms. Optionally, the kidney tissue is a piece of kidney tissue weighing between 2 mg and 100 mg. Optionally, the piece of kidney tissue weighs between 2 mg and 50 mg. Optionally, the tissue is obtained from the subject using a biopsy. Optionally, the nephroforms are introduced into the subject from which the kidney tissue was obtained. Optionally, the subject suffers from CKD. Optionally, the nephroforms form tubules upon introduction into the subject. Optionally, the tissue is subjected to mechanical and enzymatic digestion while maintaining the same weight of the kidney tissue. Optionally, the kidney tissue is not filtered or strained after mechanical and enzymatic digestion. Optionally, the tissue is washed with a buffer solution before incubating the tissue with SCM. Optionally, the cell culture plate is a 35 mm to 100 mL culture plate/tube. Optionally, the SCM comprises fetal bovine serum. Optionally, the SCM further comprises at least one growth factor selected from the group consisting of: fibroblast growth factor (FGF) stem cell factor (SCF) (5 ng/mL), and epidermal growth factor (EGF). Optionally, the kidney tissue is incubated for between 7 and 10 days in step d. Optionally, the kidney tissue is incubated for 8 days. Optionally, the medium is changed over the course of the incubation. Optionally, the medium is changed after 2 days. Optionally, the kidney tissue is incubated until cells achieve confluence. Optionally, the dissociating agent comprises trypsin or a trypsin analogue. Optionally, after a first harvesting, harvested cultured cells are reincubated with serum containing media having at least one growth factor in a second cell culture plate for between 2 and 6 days, then harvested from the second cell culture plate. Optionally, the incubation in step is for 6-7 days. Optionally, the incubation is in the presence of a growth factor. Optionally, the growth factor is selected from the group consisting of epidermal growth factor (EGF) and fibroblast growth factor (FGF). Optionally, the incubation in step is in the presence of insulin or progesterone. Optionally, the nephroforms do not attach to the surface of the container in which they are cultured, and more than 70% of the nephroform cells can be removed from the surface of the container by mechanical manipulations that do not cause significant damage to the cells.

According to an embodiment, described herein is a method for preparing cultured cells, the method comprising: obtaining kidney tissue from a human subject; mechanically dissociating the tissue; subjecting the tissue to enzymatic digestion; and incubating the tissue with media in a cell culture plate to form cultured cells. Optionally, the kidney tissue is a piece of kidney tissue weighing between 2 mg and 100 mg. Optionally, the piece of kidney tissue weighs between 2 mg and 50 mg. Optionally, the tissue is obtained from the subject using a biopsy. Optionally, the tissue is subjected to mechanical and enzymatic digestion while maintaining the same weight of the kidney tissue. Optionally, the kidney tissue is not filtered or strained after mechanical and enzymatic digestion. Optionally, the tissue is washed with a buffer solution before incubating the tissue with media. Optionally, the cell culture plate is a 35 mm to 100 mL culture plate/tube. Optionally, the media is SCM, comprising fetal bovine serum. Optionally, the SCM further comprises at least one growth factor selected from the group consisting of: fibroblast growth factor (FGF) stem cell factor (SCF) (5 ng/mL), and epidermal growth factor (EGF). Optionally, the kidney tissue is incubated for between 7 and 10 days. Optionally, kidney tissue is incubated for 8 days. Optionally, the medium is changed over the course of the incubation. Optionally, the medium is changed after 2 days. Optionally, the kidney tissue is incubated until cells achieve confluence. Optionally, the method further comprises harvesting the cultured cells from the cell culture plate using a dissociating agent. Optionally, the dissociating agent comprises trypsin or a trypsin analogue. Optionally, after a first harvesting, harvested cultured cells are reincubated with media having at least one growth factor in a second cell culture plate for between 2 and 6 days, then harvested from the second cell culture plate. Optionally, the medium is renal epithelial medium. Optionally, the renal epithelial medium comprises at least one of human Epidermal growth factor (hEGF), transferrin, and insulin.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for manufacture of cell nephroforms for kidney therapy, the method comprising:
a. obtaining kidney tissue from a human subject;
b. mechanically dissociating the tissue;
c. subjecting the tissue to enzymatic digestion, wherein the kidney tissue is not filtered or strained after mechanical and enzymatic digestion;
d. incubating the tissue with serum containing media (SCM) having at least one growth factor in a cell culture plate to form cultured cells;
e. harvesting the cultured cells from the cell culture plate using a dissociating agent;
f. reseeding the cultured cells in tissue culture flasks, and incubating the cells under non-adherent conditions, to form nephroforms.

2. The method according to claim 1 wherein the kidney tissue is a piece of kidney tissue weighing between 2 mg and 100 mg.

3. The method according to claim 2 wherein the piece of kidney tissue weighs between 2 mg and 50 mg.

4. The method according to claim 1 wherein the tissue is obtained from the subject using a biopsy.

5.-7. (canceled)

8. The method according to claim 1 wherein the tissue is subjected to mechanical and enzymatic digestion while maintaining the same weight of the kidney tissue.

9. (canceled)

10. The method according to claim 1 wherein the tissue is washed with a buffer solution before incubating the tissue with SCM.

11.-25. (canceled)

26. A method for preparing cultured cells, the method comprising:
a. obtaining kidney tissue from a human subject;
b. mechanically dissociating the tissue;
c. subjecting the tissue to enzymatic digestion; and
d. incubating the tissue with media in a cell culture plate to form cultured cells, wherein the kidney tissue is not filtered or strained after mechanical and enzymatic digestion.

27. The method according to claim 26 wherein the kidney tissue is a piece of kidney tissue weighing between 2 mg and 100 mg.

28. The method according to claim 27 wherein the piece of kidney tissue weighs between 2 mg and 50 mg.

29. The method according to claim 26 wherein the tissue is obtained from the subject using a biopsy.

30. The method according to claim 26 wherein the tissue is subjected to mechanical and enzymatic digestion while maintaining the same weight of the kidney tissue.

31. (canceled)

32. The method according to claim 26 wherein the tissue is washed with a buffer solution before incubating the tissue with media.

33. The method according to claim 26 wherein the cell culture plate is a 35 mm to 100 mL culture plate/tube.

34. The method according to claim 26 wherein the media is SCM, comprising fetal bovine serum.

35. The method according to claim 34 wherein the SCM further comprises at least one growth factor selected from the group consisting of:
fibroblast growth factor (FGF) stem cell factor (SCF) (5 ng/mL), and epidermal growth factor (EGF).

36. The method according to claim 26 wherein the kidney tissue is incubated for between 7 and 10 days in step d.

37. (canceled)

38. The method according to claim 26 wherein the medium is changed over the course of the incubation.

39. The method according to claim 38 wherein the medium is changed after 2 days.

40. The method according to any claim 26 wherein the kidney tissue is incubated until cells achieve confluence.

41. The method according to claim 26, further comprising:
e. harvesting the cultured cells from the cell culture plate using a dissociating agent.

42.-45. (canceled)
